(19) United States
(12) Patent Application Publication
(10) Pub. No.: US 2009/0281143 A1
(43) Pub. Date: Nov. 12, 2009

(54) Dose Reduction of a Cannabinoid CB1 Receptor Antagonist in the Treatment of Overweight or Obesity

(75) Inventors: Peter Literati Nagy, Jablonka (HU); Zoltan Szilvassy, Kutvolgyl utl (HU); Kalman Tory, Katona Jozsef (HU); Attila Kolonics, Bonyhadi (HU); Sandor Bernath, Buzavirag (HU); Laszlo Vigh, Kikinda (HU); Jesse Roth, Whitestone, NY (US); G. Alexander Fleming, Harper's Ferry, WV (US); Michael Brownstein, Rockville, MD (US); Janos Egri, Dereglye (HU)

Correspondence Address: FISH & RICHARDSON PC, P.O. BOX 1022, MINNEAPOLIS, MN, 55440-1022, US

(73) Assignee: N-Gene Research Laboratories, Inc.

(21) Appl. No.: 12/331,890

(22) Filed: Dec. 10, 2008

Related U.S. Application Data
(60) Provisional application No. 61/012,684, filed on Dec. 10, 2007.

(51) Int. Cl. A61K 31/4545; A61P 3/04

(52) U.S. Cl. 514/318

(57) ABSTRACT

O-(3-Piperidino-2-hydroxy-1-propyl)-nicotinic amidoxime or a pharmaceutically acceptable acid addition salt thereof is administered to patients suffering from overweight or obesity and treated with a cannabinoid CB₁ receptor antagonist such as rimonabant to reduce overweight or obesity and, preferably, reduce one or more unfavourable psychiatric side effects of the cannabinoid CB₁ receptor antagonist.

### RELATED APPLICATION INFORMATION

This application claims priority to U.S. provisional application Ser. No. 61/012,684, filed Dec. 10, 2007.

### FIELD OF THE INVENTION

This disclosure related to the use of a cannabinoid CB₁ receptor antagonist, for example rimonabant, in combination with O-(3-piperidino-2-hydroxy-1-propyl)-nicotinic amidoxime for the treatment of overweight, obesity or body weight gain. This disclosure also relates to a reduction of the psychiatric side effect of a cannabinoid CB₁ receptor antagonist when used in combination with O-(3-piperidino-2-hydroxy-1-propyl)-nicotinic amidoxime.

### BACKGROUND OF THE INVENTION

Overweight and obesity represent the most prevalent nutritional problem in the developed countries. According to the estimations of World Health Organization, more than 300 million adults are obese worldwide. In case of adults, overweight is characterized by a body mass index of 25-30 kg/m², while a body mass index of above 30 kg/m² indicates obesity.

Overweight and obesity themselves are associated with hypertension and abnormal metabolic changes such as insulin resistance and dyslipidemia which are risk factors for diabetes. Obesity (particularly abdominal obesity), insulin resistance and dyslipidemia are major features of “pre-diabetes” (metabolic syndrome) that leads to type 2 diabetes mellitus. Diabetes is accompanied by increased mortality due to a greater risk of cardiovascular diseases. Thus, it can be stated that obesity predisposes to diseases of high risk such as type 2 diabetes mellitus, cardiovascular diseases, osteoarthritis, formation of gall stones and various malignant diseases.

Cannabis binds to and expresses its effect through specific receptors named as cannabinoid receptors. Currently, there are two known subtypes of cannabinoid receptors: CB₁ and CB₂. The cannabinoid CB₁ receptors are believed to play a role in controlling food consumption, food intake, energy expenditure, the neuroendocrine response of the stress system, and the metabolic functions of crucial peripheral tissues such as the adipose tissue, the gastrointestinal tract, the liver, and the skeletal muscles. Cannabinoid receptor antagonists block or inhibit the activation of cannabinoid receptors.

Therefore, one of the approaches to reduce overweight and obesity consists in the administration of a cannabinoid CB₁ receptor antagonist that reduces the appetite. However, in the administration of cannabinoid receptor antagonists there is a risk of the occurrence of psychiatric side effects. A well known potent cannabinoid CB₁ receptor antagonist is rimonabant, i.e., N-piperidino-5-(4-chlorophenyl)-1-(2,4-dichlorophenyl)-4-methylpyrazole-3-carboxamide (European Patent No. 656 354) that is rather effective in the reduction of obesity, however, produces adverse psychiatric effects especially anxiety, depression, suicidal ideation etc. Thus, in the treatment of obese patients with rimonabant, there is a relatively high risk of psychiatric side effects. (FDA Briefing Document NDA 21-888, Zimulti (rimonabant) Tablets, 20 mg, issued by Advisory Committee, Jun. 13, 2007).

O-(3-piperidino-2-hydroxy-1-propyl)-nicotinic amidoxime (abbreviated as BGP-15) is a compound useful in the treatment of diabetic angiopathy, a complication of diabetes resulting in the damage of blood vessels. BGP-15 is described, for example, in U.S. Pat. No. 4,187,220.

U.S. Pat. No. 6,306,878 refers to a method for the protection of the mitochondrial genome and/or mitochondrion from damage leading to myopathies and neurodegenerative diseases which comprises administering an effective non-toxic dose to a patient susceptible to such damage of an amidoximic acid derivative including BGP-15. A preferred myopathy is cardiomyopathy. Neurodegenerative diseases include Parkinson's disease, Huntington's disease and amyotrophic lateral sclerosis.

U.S. Pat. No. 6,458,371 refers to a composition comprising 0.1-30% of a hydroximic acid derivative including BGP-15 as the active ingredient and a carrier that is in the form of a cream, lotion, foam or spray. The composition is suitable for reducing the incidence of photodamage by radiation with UV-B.

U.S. Pat. No. 6,884,424 refers to a method for preventing actinic keratosis by applying a hydroximic acid derivative including BGP-15 to the affected skin surface.

U.S. Pat. No. 6,451,851 refers to a method of treating a patient suffering from a viral infection comprising administering to the patient a pharmaceutically effective amount of a known antivirally active agent together with a hydroximic acid derivative including BGP-15.

U.S. Pat. No. 6,440,998 refers to a pharmaceutical composition having antitumor activity with reduced side effect comprising cisplatin or carboplatin and a hydroximic acid derivative including BGP-15. U.S. Pat. No. 6,656,955 refers to a pharmaceutical composition having antitumor activity with reduced side effect comprising paclitaxel or docetaxel and a hydroximic acid derivative including BGP-15. U.S. Pat. No. 6,720,337 refers to a pharmaceutical composition having antitumor activity with reduced side effect comprising oxaliplatin and a hydroximic acid derivative including BGP-15. U.S. Pat. No. 6,838,469 refers to a pharmaceutical composition having antitumor activity with reduced side effect comprising pyrimidine derivatives and BGP-15.

WO 00/07580 discloses experimental data for the antidiabetic effect of BGP-15 in the treatment of type 1 diabetes mellitus. It is to be noted that type 1 diabetes mellitus is an autoimmune disease occurring at young age, while type 2 diabetes mellitus is a metabolic disease occurring at higher age.

WO 03/007951 refers to a pharmaceutical combination of hydroximic acid derivatives including BGP-15 and an antidiabetic or anti-hyperlipidemic active agent for the prevention or treatment of a prediabetic state, metabolic X-syndrome or diabetes mellitus as well as disorders which are associated with the states listed above, namely endogenic metabolic disorders, insulin resistance, dislipidemia, alopecia, diffuse effluvium and/or female endocrine disorders based on androgenic preponderance. In the description, laboratory data indicate that BGP-15 enhances, synergistically, the effect of the known antidiabetic agent metformin and troglitazone, respectively. The laboratory data also show that BGP-15 in itself enhances the insulin sensitivity (thus, reduces the insulin resistance) in both normal and hyper-cholesterolemic animals relative to the control.

WO 2005/122678 refers to the use of BGP-15 in a pharmaceutical composition having prokinetic effect (i.e. induces activity in the stomach and intestines. Prokinetic effect includes possible treatment of reflux esophagitis, gastroparesis, influencing bile flow from the gall bladder etc.

WO 2005/123049 refers to the use of BGP-15 for mitochondrial genesis i.e. to increase the number of mitochondria in the cells resulting in a roborating effect.

WO 2006/079910 refers to the use of BGP-15 for the treatment of lesions in the oral cavity, especially periodontal disease.

### SUMMARY OF THE INVENTION

It has been found that O-(3-piperidino-2-hydroxypropyl)-nicotinic amidoxime or a pharmaceutically suitable acid addition salt thereof can be used for increasing the effect of cannabinoid CB₁ antagonists, especially rimonabant, synergistically. Due to the dose reduction of the cannabinoid CB₁ antagonists in the treatment of overweight or obesity, the psychiatric side effects that occur in the treatment with cannabinoid CB₁ antagonists, especially rimonabant, can be reduced by the simultaneous administration of O-(3-piperidino-2-hydroxypropyl)-nicotinic amidoxime or a pharmaceutically suitable acid addition salt thereof.

Described herein is a method for the treatment of overweight or obesity in a patient requiring a treatment with a cannabinoid CB₁ receptor antagonist comprising administering an effective amount of a known cannabinoid CB₁ receptor antagonist and an effective non-toxic amount of O-(3-piperidino-2-hydroxy-1-propyl)-nicotinic amidoxime or a pharmaceutically acceptable acid addition salt thereof to the patient, wherein the combined administration of the O-(3-piperidino-2-hydroxy-1-propyl)-nicotinic amidoxime or a pharmaceutically acceptable acid addition salt thereof and the cannabinoid CB₁ receptor antagonist produces synergistic effect.

Also described is a method for the treatment of overweight or obesity in a patient requiring a treatment with a cannabinoid CB₁ receptor antagonist having unfavourable psychiatric side effect comprising administering an effective amount of a known cannabinoid CB₁ receptor antagonist and an effective non-toxic amount of O-(3-piperidino-2-hydroxy-1-propyl)-nicotinic amidoxime or a pharmaceutically acceptable acid addition salt thereof to the patient, wherein the administration of the O-(3-piperidino-2-hydroxy-1-propyl)-nicotinic amidoxime or a pharmaceutically acceptable acid addition salt thereof reduces the psychiatric side effect.

Also described is a pharmaceutical composition comprising a known cannabinoid CB₁ receptor antagonist and O-(3-piperidino-2-hydroxy-1-propyl)-nicotinic amidoxime or a pharmaceutically acceptable acid addition salt thereof in admixture with one or more conventional carrier(s).

Also described is a pharmaceutical composition for the treatment of overweight or obesity comprising a known cannabinoid CB₁ receptor antagonist and O-(3-piperidino-2-hydroxy-1-propyl)-nicotinic amidoxime or a pharmaceutically acceptable acid addition salt thereof in admixture with one or more conventional carrier(s). In some cases, the pharmaceutical composition is a unit dosage from. In some cases, the pharmaceutical composition contains a mixture of cannabinoid CB₁ receptor antagonist and O-(3-piperidino-2-hydroxy-1-propyl)-nicotinic amidoxime effective for treatment of overweight or obesity, but has reduced side-effects from the cannabinoid CB₁ receptor antagonist compared to an amount of cannabinoid CB₁ receptor antagonist effective for treatment of overweight or obesity.

Also described is a pharmaceutical composition for the treatment of overweight or obesity and having reduced psychiatric side effect comprising a known cannabinoid CB₁ receptor antagonist and O-(3-piperidino-2-hydroxy-1-propyl)-nicotinic amidoxime or a pharmaceutically acceptable acid addition salt thereof in admixture with one or more conventional carrier(s).

Also described is a method for treating a patient being treated with a known cannabinoid CB₁ receptor antagonist, the method comprising administering to the patient being treated with a known cannabinoid CB₁ receptor antagonist, a composition comprising O-(3-piperidino-2-hydroxy-1-propyl)-nicotinic amidoxime or a pharmaceutically acceptable acid addition salt thereof to the patient.

In various embodiments the cannabinoid CB₁ receptor antagonist is preferably rimonabant or a pharmaceutically acceptable acid addition salt and/or solvate or hydrate thereof.

In various embodiments the psychiatric side effects comprise, in particular, anxiety, depression and suicidal ideation.

The details of one or more embodiments are set forth in the description below. Other features, objects, and advantages of the invention will be apparent from the description, and from the claims.

### DESCRIPTION OF PREFERRED EMBODIMENTS

The cannabinoid CB₁ receptor antagonist includes any known active agent that antagonizes the cannabinoid CB₁ receptor. Preferred cannabinoid CB₁ receptor antagonists include N-piperidino-5-(4-chlorophenyl)-1-(2,4-dichlorophenyl)-4-methylpyrazole-3-carboxamide (rimonabant) and N′-piperidino-5-(4-bromophenyl)-1-(2,4-dichlorophenyl)-4-ethylpyrazole-3-carboxamide or a pharmaceutically suitable acid addition salt thereof or a solvate of the base or a solvate of the acid addition salt.

A pharmaceutically suitable acid addition salt is a salt formed with an inorganic acid such as hydrochloric acid, sulfuric acid etc. or with an organic acid such as acetic acid, lactic acid, tartaric acid etc. Preferred acid addition salts include hydrochlorides, acetates, maleates etc. A preferred acid addition salt of O-(3-piperidino-2-hydroxy-1-propyl)nicotinic amidoxime is the dihydrochloride thereof. BGP-15 can be prepared by the process described in, e.g., U.S. Pat. No. 4,187,220.

In one embodiment, a conventional dose of a known cannabinoid CB₁ receptor antagonist, preferably rimonabant, is administered to a patient requiring treatment of overweight or obesity, and, simultaneously, a dose of BGP-15 or a pharmaceutically suitable acid addition salt thereof is administered. This non-toxic dose of BGP-15 increases the effect of the cannabinoid CB₁ receptor antagonist synergistically, and reduces, effectively, the psychotic side effect associated with the administration of the cannabinoid CB₁ receptor antagonist.

In some embodiments, the known cannabinoid CB₁ receptor antagonist such as rimonabant is not administered simultaneously with BGP-15. Thus, while the two active agents in the combination therapy, e.g. rimonabant and BGP-15, can be administered simultaneously, they need not be. For example, administration of a first active agent can precede the administration of a second active agent by minutes, hours, days or weeks. Thus the two active agents can be administered within minutes of each other or within several hours of each other or several days of each other or several weeks of each other. In some cases, even longer intervals are possible. While in many cases it is desirable that the two active agents used in a combination therapy be present in the patient's body at the same time, this need not be so. Combination therapy can also include two or more administrations of one of the active agents or both active agents used in the combination.

Combination therapy can also include the administration of the two active agents via different routes or locations. For example, One active agent is administered orally and the other active agent is administered parenterally or one active agent is administered orally and the other active agent is administered locally. In each case, the active agents can be administered either simultaneously or sequentially.

Generally, the daily dose of the known cannabinoid CB₁ receptor antagonist, preferably rimonabant, for an adult person of about 70 kg body weight amounts to 1-1000 mg (0.014 mg/k-14 mg/kg), preferably 1-100 mg (0.014 mg/k-1.4 mg/kg), in general, 2-20 mg (0.028 mg/k-0.28 mg/kg). The similar daily dose of BGP-15 (as dihydrochloride) is, in general, 5-1000 mg (0.071-14 mg/kg), preferably 50-500 mg (0.714-14 mg/kg).

According to an especially preferred method of the invention, 5-20 mg of rimonabant and 50-500 mg of BGP-15 dihydrochloride are administered to an adult, daily.

In case of the pharmaceutical composition of the invention each of the two active agents (i.e. the known cannabinoid CB₁ receptor antagonist and BGP-15) has been converted, one by one, to separate pharmaceutical compositions using one or more conventional carrier(s) and any of the usual processes of drug manufacture, and in this case the two sorts of pharmaceutical composition obtained are administered to the patient simultaneously or one after the other; or the two active agents have been converted to one single pharmaceutical composition that can be administered to the patient being in need thereof. In the latter case, the pharmaceutical composition may contain a mixture of the two active agents, or each of the active agents may be present at a different site in the pharmaceutical composition, e.g. one of them in the tablet core and the other in a coating of the tablet core. Of course, one or more conventional carriers and any of the usual processes of drug manufacture are used to prepare this single pharmaceutical composition.

The pharmaceutical composition of the invention contains an effective non-toxic amount of a known cannabinoid CB₁ receptor antagonist, preferably rimonabant, or a pharmaceutically suitable acid addition salt and/or a solvate thereof and an effective non-toxic amount of BGP-15 or a pharmaceutically suitable acid addition salt thereof in addition to one or more pharmaceutically acceptable carrier(s). The pharmaceutical composition may include any dosage form suitable for peroral, parenteral, transdermal or rectal administration or for local treatment, and can be solid or liquid.

The solid pharmaceutical compositions suitable for peroral administration may be powders, capsules, tablets, film-coated tablets, microcapsules etc., and can comprise binding agents such as gelatine, sorbitol, poly(vinylpyrrolidone) etc.; filling agents such as lactose, glucose, starch, calcium phosphate etc.; auxiliary substances for tabletting such as magnesium stearate, talc, poly(ethylene glycol), silica etc.; wetting agents such as sodium laurylsulfate etc. as the carrier.

The liquid pharmaceutical compositions suitable for peroral administration may be solutions, suspensions or emulsions and can comprise e.g. suspending agents such as gelatine, carboxymethylcellulose etc.; emulsifiers such as sorbitane monooleate etc.; solvents such as water, oils, glycerol, propylene glycol, ethanol etc.; preservatives such as methyl p-hydroxybenzoate etc. as the carrier.

Pharmaceutical compositions suitable for parenteral administration consist of sterile solutions of the active ingredients, in general.

Dosage forms listed above as well as other dosage forms are known per se, see e.g. Remington's Pharmaceutical Sciences, Mack Publishing Co., Easton, USA.

The pharmaceutical composition contains dosage unit, in general. The daily dose can be administered in one or more portions. The actual dosage depends on many factors and is determined by the doctor.

The pharmaceutical composition is prepared by admixing the active ingredients to one or more carrier(s), and converting the mixture obtained to a pharmaceutical composition in a manner known per se. Useful methods are known from the literature, e.g. Remington's Pharmaceutical Sciences mentioned above.

A preferred pharmaceutical composition of the invention contains a known cannabinoid CB₁ receptor antagonist selected from the group consisting of rimonabant and N-piperidino-5-(4-bromophenyl)-1-(2,4-dichlorophenyl)-4-ethylpyrazole-3-carboxamide or a pharmaceutically acceptable acid addition salt and/or a solvate thereof in addition to BGP-15 or a pharmaceutically suitable acid addition salt thereof, preferably BGP-15 dihydrochloride.

### Example

### Inhibition of Body Weight Gain in High Fat Diet Exposed Mice by Rimonabant and BGP-15

OF-1 female mice weighing about 18-20 g at the beginning of the experiment were used in the study. A group of 9 mice were kept on standard laboratory chow, while the other experimental groups were exposed to high fat diet and to daily oral treatment with the following compounds: vehicle, rimonabant 2 mg/kg, rimonabant 10 mg/kg, BGP-15 dihydrochloride 20 mg/kg and rimonabant 2 mg/kg+BGP-15 dihydrochloride 20 mg/kg. The high fat diet consisted of palatable food that contained 50% fat. The group on standard laboratory chow was also treated with vehicle. Treatment was performed with all of the drugs, orally, at 5 ppm. The weight of the animals was measured weekly. The body weight gain (BWG) data at the end of the second and third week are shown in Table 1.

TABLE 1

| | Body weight gain in g | |
|---|---|---|
| Group | after 2 weeks | after 3 weeks |
| Standard laboratory chaw + vehicle | 8.7 | 10.6 |
| (control) | | |
| High fat diet (HFD) + vehicle | 12.6 | 15.9 |
| (control_{HFD}) | | |
| HFD + rimonabant 2 mg/kg | 10.6 | 14.1 |
| HFD + rimonabant 10 mg/kg | 10.2 | 13.0 |
| HFD + BGP-15 dihydrochloride 20 mg/kg | 10.4 | 13.6 |
| HFD + rimonabant 2 mg/kg + | 9.4 | 11.8 |
| BGP-15 dihydrochloride 20 mg/kg | | |

From Table 1 it can be seen that, in the control group, high fat diet caused a body weight gain of 44% after 2 weeks, and 50% after 3 weeks, in relation to the weight gain in the control group in which the animals were fed with standard laboratory chow. Consequently, the high fat diet produced obese mice. In the test group treated with high fat diet and 2 mg/kg of rimonabant, the weight gain was 22% after 2 weeks, and 33% after 3 weeks, in relation to that of the control group fed with standard laboratory chaw. In the test group treated with high fat diet and 10 mg/kg of rimonabant, a weight gain of 17% after 2 weeks, and 22.6% after 3 weeks was obtained, in relation to that of the control group fed with standard laboratory chow. In the test group treated with high fat diet and 20 mg/kg of BGP-15 dihydrochloride, the weight gain was 19.5% after 2 weeks, and 28.3% after 3 weeks, in relation to that of the control group fed with standard laboratory chaw. Thus, it can be stated that neither rimonabant nor BGP-15, alone, could inhibit the weight gain sufficiently to compensate the effect of high fat diet in the test groups.

However, in the test group treated with both BGP-15 dihydrochloride and the lower dose of rimonabant, the weight gain was as low as 8% after 2 weeks, and 11.3% after 3 weeks, in relation to that of the control group fed with standard laboratory chaw. Thus, it is evident, that the weight gain produced by a high fat diet can be compensated by a combined treatment with a lower dose of rimonabant and with BGP-15 dihydrochloride within about 10%.

Consequently, BGP-15 produces synergism with rimonabant regarding the weight reducing effect. Since, in the method of the invention, it is sufficient to administer a lower dose of rimonabant in the treatment of overweight and obesity, a lower incidence of the unfavourable psychiatric side effect of rimonabant can be expected.

What is claimed:

1. A method for overweight or obesity in a patient comprising administering an effective amount of a cannabinoid CB₁ receptor antagonist and an effective amount of O-(3-piperidino-2-hydroxy-1-propyl)-nicotinic amidoxime or a pharmaceutically acceptable acid addition salt thereof to the patient, wherein the combined administration of the O-(3-piperidino-2-hydroxy-1-propyl)-nicotinic amidoxime or a pharmaceutically acceptable acid addition salt thereof.

2. A method for overweight or obesity in a patient comprising administering an effective amount of a cannabinoid CB₁ receptor antagonist and an effective amount of O-(3-piperidino-2-hydroxy-1-propyl)-nicotinic amidoxime or a pharmaceutically acceptable acid addition salt thereof to the patient, wherein the combined administration of the O-(3-piperidino-2-hydroxy-1-propyl)-nicotinic amidoxime or a pharmaceutically acceptable acid addition salt thereof, wherein the administration of the O-(3-piperidino-2-hydroxy-1-propyl)-nicotinic amidoxime or a pharmaceutically acceptable acid addition salt thereof reduces the psychiatric side effect.

3. The method of claim 2 in which the psychiatric side effect is anxiety.

4. The method of claim 2 in which the psychiatric side effect is depression.

5. The method of claim 1 or claim 2 in which the cannabinoid CB₁ receptor antagonist is rimonabant or a pharmaceutically acceptable acid addition salt and/or a solvate thereof.

6. The method of claim 1 or claim 2 in which the cannabinoid CB₁ receptor antagonist is N-piperidino-5-(4-bromophenyl)-1-(2,4-dichlorophenyl)-4-ethylpyrazole-3-carboxamide or a pharmaceutically acceptable acid addition salt and/or a solvate thereof.

7. The method of any of claim 1 or claim 2 in which O-(3-piperidino-2-hydroxy-1-propyl)-nicotinic amidoxime dihydrochloride is administered.

8. A pharmaceutical composition comprising a cannabinoid CB₁ receptor antagonist and O-(3-piperidino-2-hydroxy-1-propyl)-nicotinic amidoxime or a pharmaceutically acceptable acid addition salt thereof in admixture with one or more conventional carriers.

9. The pharmaceutical composition of any of claim 8 comprising rimonabant or a pharmaceutically acceptable acid addition salt and/or a solvate thereof and O-(3-piperidino-2-hydroxy-1-propyl)-nicotinic amidoxime or a pharmaceutically acceptable acid addition salt thereof.

10. A pharmaceutical composition comprising N-piperidino-5-(4-bromophenyl)-1-(2,4-dichlorophenyl)-4-ethylpyrazole-3-carboxamide or a pharmaceutically acceptable acid addition salt and/or a solvate thereof and O-(3-piperidino-2-hydroxy-1-propyl)-nicotinic amidoxime or a pharmaceutically acceptable acid addition salt thereof.

11. A method for treating a patient being treated with a cannabinoid CB₁ receptor antagonist, the method comprising administering to the patient being treated with a cannabinoid CB₁ receptor antagonist, a composition comprising O-(3-piperidino-2-hydroxy-1-propyl)-nicotinic amidoxime or a pharmaceutically acceptable acid addition salt thereof to the patient.

12. A method for the treatment of overweight or obesity in a patient requiring a treatment with a cannabinoid CB₁ receptor antagonist comprising administering an effective amount of a known cannabinoid CB₁ receptor antagonist and an effective non-toxic amount of O-(3-piperidino-2-hydroxy-1-propyl)-nicotinic amidoxime or a pharmaceutically acceptable acid addition salt thereof to the patient, wherein the combined administration of the O-(3-piperidino-2-hydroxy-1-propyl)-nicotinic amidoxime or a pharmaceutically acceptable acid addition salt thereof and the cannabinoid CB₁ receptor antagonist produces synergistic effect.

13. A method for the treatment of overweight or obesity in a patient requiring a treatment with a cannabinoid CB₁ receptor antagonist having unfavourable psychiatric side effect comprising administering an effective amount of a known cannabinoid CB₁ receptor antagonist and an effective non-toxic amount of O-(3-piperidino-2-hydroxy-1-propyl)-nicotinic amidoxime or a pharmaceutically acceptable acid addition salt thereof to the patient, wherein the administration of the O-(3-piperidino-2-hydroxy-1-propyl)-nicotinic amidoxime or a pharmaceutically acceptable acid addition salt thereof reduces the psychiatric side effect.
